(19) United States
(12) Patent Application Publication
(10) Pub. No.: US 2011/0152545 A1
(43) Pub. Date: Jun. 23, 2011

(54) Process for manufacturing chlorohydrins, epodixes, diols, diols derivatives or epoxy derivatives

(75) Inventors: Dominique Balthasart, Brussels (BE); Andre Daene, Waterloo (BE)

(73) Assignee: SOLVAY SA, Brussels (BE)

(21) Appl. No.: 13/060,421

(22) PCT Filed: Sep. 7, 2009
(86) PCT No.: PCT/EP2009/061546

§ 371 Date: Feb. 23, 2011

(30) Foreign Application Priority Data
Sep. 10, 2008 (FR) 0856059

(51) Int. Cl. C07D 301/02; C07C 31/34; B01J 19/00

(52) U.S. Cl. 549/518; 568/844; 422/198

(57) ABSTRACT

Process for manufacturing a chemical: chlorohydrins, epoxides, diols, diols derivatives, epoxy derivatives or mixtures thereof, in an industrial plant, comprising a step of chemical reaction for manufacturing the chemical and at least one step selected from the group consisting of storage, supply, removal, transfer, chemical treatment and physical treatment of compounds used or produced in such process, in which at least one aqueous effluent is generated, this aqueous effluent containing at least one of overhead waters, surface waters, ground waters, waters from drinking water distribution networks, waters from industrial water distribution networks, plant cleaning waters, or waters used for heating and cooling the plant, this aqueous effluent containing at least one compound other than water and exhibiting at least one of the following features, a water solubility at 25° C. greater than or equal to 0.01 g/kg of water and a boiling point at 1013 mbar, greater than or equal to −100° C., in which at least one part of the generated aqueous effluent is collected, and in which at least one portion of the collected aqueous effluent is recycled in the manufacturing process.

The present patent application claims the benefit of the following patent application FR 0856059 filed on 10 Sep. 2008, the content of which is incorporated here by reference.

The present invention relates to a process for manufacturing a chemical.

Processes for manufacturing chemicals generate aqueous effluents that are weakly contaminated by various types of compounds. These effluents are not very suitable for biological and/or physicochemical purification treatments, before their release into the environment.

The International application WO 2006/106153 in the name of SOLVAY SA discloses an integrated process for manufacturing a chlorohydrin and an epoxide. Waters obtained from the processes, contaminated by organic substances, and the invert water from the plants are re-used in specific units of the plants. Such waters are not weakly contaminated aqueous effluents.

The objective of the invention is to supply a solution to this problem by providing a process for manufacturing a chemical chosen from the group consisting of chlorohydrins, epoxides, diols, diols derivatives, epoxy derivatives and mixtures of at least two of them, in an industrial plant, the process comprising a step of chemical reaction for manufacturing the chemical and at least one step selected from the group consisting of the steps of storage, supply, removal, transfer, chemical treatment and physical treatment of compounds used or produced in the process for manufacturing the chemical, in which at least one aqueous effluent is generated, this aqueous effluent containing at least one selected from the group consisting of overhead waters, surface waters, ground waters, waters from drinking water distribution networks, waters from industrial water distribution networks, plant cleaning waters, waters used for heating and cooling the plants, and this aqueous effluent containing at least one compound other than water, said compound exhibiting at least one of the following features, a water solubility at 25° C. greater than or equal to 0.01 g/kg of water and a boiling point at 1013 mbar, greater than or equal to −100° C., in which at least one part of said generated aqueous effluent is collected, and in which at least one portion of said collected aqueous effluent is recycled in said manufacturing process.

One of the main features of the process lies in the recycling of said collected aqueous effluent in the process for manufacturing the chemical according to the invention.

It has been found that recycling said aqueous effluent in the manufacturing process, rather than treating them and releasing them into the environment, makes it possible:
1. to avoid an energy-intensive treatment operation of said aqueous effluent, for example via evaporation, when said waters are contaminated;
2. to re-use compounds contained in said aqueous effluent, although such compounds might be present in low concentration in said water;
3. to contribute to a reduction in the overall water consumption of the process, which is particularly important on sites where the water supply is limited and/or expensive; and
4. to make the process independent of the existence, on the industrial site, of a biological water treatment plant.

Without wishing to be tied to one theoretical explanation, it is believed that, due to their origin, said waters are weakly contaminated by compounds. These compounds can be organic compounds, inorganic compounds, or mixture thereof. They are usually heavy compounds. They can be water-soluble compounds. They can be toxic, even at very low concentrations. These compounds can also exhibit several of such properties. The dilution of the compounds, especially of organic type, and possibly their toxicity, makes said waters unsuitable for a biological digestion. This high dilution of the compounds, allied to their not very volatile nature, makes their recovery via simple operations such as stripping, not very profitable.

In the process according to the invention, the amount of generated aqueous effluents that are collected is generally greater than or equal to 5% by weight of the generated aqueous effluents, usually greater than or equal to 10% by weight, commonly greater than or equal to 20% by weight, in a lot of cases greater than or equal to 30% by weight, often greater than or equal to 50% by weight, frequently greater than or equal to 70% by weight and particularly greater than or equal to 90% by weight. This amount is generally less than or equal to 99.9% by weight.

In the process according to the invention, the amount of said collected aqueous effluent that is recycled in the process for manufacturing the chemical is generally greater than or equal to 5% by weight of said collected aqueous effluent, usually greater than or equal to 10% by weight, commonly greater than or equal to 20% by weight, in a lot of cases greater than or equal to 30% by weight, often greater than or equal to 50% by weight, frequently greater than or equal to 70% by weight and particularly greater than or equal to 90% by weight. This amount is generally less than or equal to 99.9% by weight.

In the process according to the invention, the overhead waters may be chosen from the group consisting of rain water, snow, hail and mixtures of at least two of them.

In the process according to the invention, the surface waters are chosen from the group consisting of water from land ice, water from sea ice, water from snow on the surface of the ground, water from lakes, from ponds, from pools, from streams, from rivers, from brooks, from biological treatment plants, sea waters, ocean waters, and mixtures of at least two of them.

In the process according to the invention, the surface waters are often chosen from the group consisting of water from land ice or sea ice, snow on the surface of the ground, water from lakes, from ponds, from pools, from streams, from rivers, from brooks, from biological water treatment plants, and mixtures of at least two of them. The biological water treatment plant is preferably not part of the plant for carrying out the process according to the invention.

In the process according to the invention, the surface waters are frequently chosen from the group consisting of sea waters, ocean waters, and mixtures thereof.

In the process according to the invention, the ground waters may be chosen from the group consisting of waters from springs, from water tables, from underground streams, and mixtures of at least two of them.

In the process according to the invention, the waters from drinking water distribution networks may originate from the group consisting of overhead waters, surface waters and ground waters as defined above which may have undergone treatments intended to make them suitable for use in food, such as potabilization and/or desalination treatments, and mixtures of at least two of them.

In the process according to the invention, the waters from industrial water distribution networks may originate from the group consisting of overhead, surface waters and ground waters as defined above which may have undergone treatments intended to make them suitable for industrial use, such as precipitation, filtration and pH adjustment treatments, for example, and mixtures of at least two of them.

In the process according to the invention, the plant cleaning waters may be chosen from the group consisting of overhead waters, surface waters, ground waters, waters from drinking water distribution networks, waters from industrial water distribution networks, as described above, and mixtures of at least two of them, used for cleaning the plant.

In the process according to the invention, the water used for heating and/or cooling the plants may be chosen from the group consisting of overhead waters, surface waters, ground waters, waters from drinking water distribution networks, waters from industrial water distribution networks, as described above, and mixtures of at least two of them. Such waters may have undergone treatments intended to make them suitable for heating and/or cooling plants, such as addition of corrosion inhibitors, for example.

Process water like waters obtained from distillation operations for instance, demineralized water obtained from ion-exchange resins, distilled water, and water arising from steam condensation are not considered to be overhead waters, surface waters, ground waters, waters from drinking water distribution networks, waters from industrial water distribution networks, plant cleaning waters, waters used for heating and cooling the plants.

In the process according to the invention, the waters may be in the liquid state, the gas state, the solid state, or in a combination of at least two of these states. These waters are often in the liquid state and frequently in a combination of the liquid state and of the gas state.

In a first variant of the process according to the invention, said waters, before collection, have been in contact with the walls of the industrial plant in which the manufacturing process according to the invention is carried out.

In the process according to the invention, the expression “walls of the plant” is understood to mean any surface of an equipment of the plant which:
when this equipment is out of operation, can be the inner as well as the outer wall of the equipment,
when this equipment is in normal operation, can only be the surface which is not intended to be in contact with the chemical substances that take part in the process for manufacturing the chemical carried out in the plant, like for instance the external wall of the reactor, of pipes, of columns, etc.

The aforementioned chemical substances are in particular the raw materials used (reactants, catalysts, diluent, solvent), the intermediates, the products and the by-products formed in the manufacturing process.

In the process according to the invention, these walls of the plant may be external or internal walls, more particularly external or internal walls of equipment that constitutes the plant. The term “equipment” is understood to mean the vessels where chemical substances are stored and chemical reactions and/or physical operations are carried out, the pipework and couplings that connect these vessels, the parts that provide sealing at the couplings, the instruments needed for transferring chemical substances between the vessels, the instruments and devices for measuring the various parameters needed for controlling the storage, for transferring compounds and for carrying out chemical reactions and physical operations.

One example of a wall of the plant according to the invention is the outer wall of the reactor in which the manufacturing process according to the invention is carried out. Another example of such a surface is the inner wall of a heat exchanger tube located in a reactor, and in which water and/or water vapour flows. Still another example of a wall of the plant according to the present invention is the inner wall of a reactor in which the manufacturing process according to the invention was carried out, and that has been subjected to a cleaning operation with these waters during a shutdown in the operation of the reactor.

In a second variant of the process according to the invention, said waters have not been in contact with the walls of the industrial plant in which the manufacturing process according to the invention is carried out. Rain water collected without having been in contact with the walls of the plant is an example of such water.

The process for manufacturing a chemical according to the invention comprises all the steps that make it possible to pass from the reactants to the reaction products. These steps comprise, inter alia, the step of chemical reaction for manufacturing the chemical, the steps of storage, supply, removal, transfer, chemical treatment or physical treatment of compounds used or produced in the process for manufacturing the chemicals.

Among the storage steps, mention may be made, for example, of the storage of reactants before use, the storage of purges before treatment, the storage of products, the storage of an optional catalyst and of its preparations.

Among the chemical treatment steps, mention may be made, for example, of a treatment intended to recover an optional catalyst and a treatment for dissolving said catalyst.

Among the physical treatment steps, mention may be made, for example, of the operations for separation via stripping, distillation, evaporation, extraction, settling, and filtration, heat exchange, heating and cooling operations.

Among the supply, removal or transfer steps, mention may be made, for example, of the operations of recycling, purging and discharging effluents, the transport of fluids between the various pieces of equipment in which the chemical reactions, storage and chemical and physical treatments take place.

The process for manufacturing a chemical according to the invention may be carried out in a continuous or discontinuous mode. That process is often carried out in the continuous mode.

Any mode of contact between the waters and the walls of the plant can be envisaged. For rain water, it may for example be runoff of rain on the plant and/or a runoff by spattering from puddles of rain water formed in the vicinity of the plant. It may also be a contact of the waters during cleaning of the plant. Another example is the contact between the water and/or the steam circulating in the pipework, used for heating and cooling the plants.

In the process for manufacturing a chemical according to the invention, the chemical is chosen from the group consisting of chlorohydrins, epoxides, diols, diols derivatives, epoxy derivatives and mixtures of at least two of them.

In the process according to the invention, the chlorohydrins are usually chosen from mono chloro ethanol, mono chloropropanol, monochloropropanediol, dichloropropanol, and mixtures of at least two of them. Monochloropropanediol, dichloropropanol, and mixtures thereof are often encountered. Dichloropropanol is frequently encountered.

In the process according to the invention, the epoxides are usually chosen from the group consisting of ethylene oxide, propylene oxide, epichlorohydrin, glycidol or mixtures of at least two of them. Epichlorohydrin is frequently encountered.

In the process according to the invention, the diols are generally chosen from the group consisting of ethylene glycol, 1,2-propanediol, 1,3-propanediol, monochloropropanediol, and mixtures of at least two of them. Monochloropropanediol is often encountered.

In the process according to the invention, the diols derivatives may be chosen from the group consisting of ethers and esters of diols, in particular those of 1,2-propanediol and of 1,3-propanediol, and mixtures thereof.

In the process according to the invention, the epoxy derivatives are usually chosen from the group consisting of epoxy resins, glycidyl ethers, glycidyl esters, glycidyl amides, glycidyl imides, glycidyl amines, products that can be used as coagulants, wet-strength resins, cationization agents, flame retardants, ingredients for detergents, epichlorohydrin elastomers, halogenated polyethers-polyols, monochloropropanediol, and mixture of at least two of them.

In the process according to the invention, mixtures of chlorohydrins and epoxides may be encountered. Diols are frequently encountered. Epoxy derivatives are often encountered.

In the process according to the invention, when the chemical is a chlorohydrin, this may be obtained by any process. This process may be chosen from the group consisting of hypochlorination of olefins, chlorination of unsaturated aliphatic alcohols, hydrochlorination of polyhydroxylated aliphatic hydrocarbons, and any combination of at least two of them. Processes for the hydrochlorination of polyhydroxylated aliphatic hydrocarbons are often encountered. The process for manufacturing dichloropropanol by subjecting glycerol to a reaction with a chlorination agent, preferably hydrogen chloride, is of particular interest.

In the process according to the invention, when the chemical is an epoxide, this may be obtained by any process. This process may be chosen from the group consisting of epoxidation of olefins, dehydrochlorination of chlorohydrins and combinations thereof. Processes for the dehydrochlorination of chlorohydrins are often encountered. Processes for dehydrochlorination by reaction between a chlorohydrin and a basic agent are frequently encountered. The process for manufacturing epichlorohydrin by subjecting dichloropropanol to a reaction with a basic agent if of particular interest.

In the process according to the invention, when the chemical is a diol, this may be obtained by any process. This process may be chosen from the group consisting of hydroxylation of olefins using peracids, hydrolysis of epoxides, hydrogenation of β-hydroxyaldehydes, of unsaturated 1,4-diols, of diacids, of diesters, of phenols, and of phenol derivatives, fermentation of biomass, hydrogenation of sugars, hydrogenolysis of sugar derivatives such as sorbitol and xylitol, and any combination of at least two of them.

In the process according to the invention, when the chemical is an epoxy derivative, this may be obtained by any process, such as described in Application WO 2008/152045 in the name of SOLVAY SA, the content of which is hereby incorporated by reference, more specifically the passages from page 32, line 6, to page 63, line 4, and in Application WO 2008/152044 in the name of SOLVAY SA, the content of which is hereby incorporated by reference, more specifically the passages from page 13, line 22, to page 44, line 8, and in Application PCT/EP2009/053766, in the name of SOLVAY SA, the content of which is hereby incorporated by reference, more specifically the passages from page 27, line 10, to page 33, line 7.

Of particular interest are processes wherein epichlorohydrin is subjected to a reaction with at least one compound chosen from monoalcohols, monocarboxylic acids, polyols, polyamines, amino alcohols, polyimides, polyamides, polycarboxylic acids, ammonia, amines, polyaminoamides, polyimines, amine salts, phosphoric acid, phosphoric acid salts, phosphorus oxychlorides, phosphoric acid esters, phosphonic acids, esters of phosphonic acids, salts of phosphonic acids, phosphinic acids, esters of phosphinic acids, salts of phosphinic acids, phosphine oxides, phosphines, ethoxylated alcohols, alkylene or phenylene oxides, and mixtures of at least two of these compounds, or in which the epichlorohydrin is subjected to a homopolymerization reaction, or in which epichlorohydrin is subjected to a reaction of oligomerisation, of co-oligomerisation, of condensation, of dehydro chlorination and of hydrolysis, with water, or with a di- or polyhydroxylated compound which may optionally be halogenated and/or have ether oxide bonds and/or double bonds capable of being halogenated in a subsequent stage, or wherein epichlorohydrin is subjected to a reaction with water.

Of more particular interest are processes wherein epichlorohydrin is subjected to a reaction:
with at least one compound containing at least one active hydrogen atom, so as to obtain an epoxy derivative, selected from the group consisting of an epoxy resin, a glycidyl ether, a glycidyl ester, a glycidyl amide, a glycidyl imide or a mixture of at least two of them; or
with ammonia, an amine, a polyaminoamide or a polyimine, so as to obtain a coagulant; or
with a polyamine, a polyamide or a polyaminoamide, so as to obtain a wet-strength resin for the treatment of paper; or
with an amine or an amine salt, so as to obtain a cationizing agent; or
with a compound chosen from phosphoric acid, a phosphoric acid salt, a phosphorus oxychloride, a phosphoric acid ester, a phosphonic acid, a phosphonic acid ester, a phosphonic acid salt, a phosphinic acid, a phosphinic acid ester, a phosphinic acid salt, a phosphine oxide or a phosphine, so as to obtain a flame retardant; or
with a monoalcohol containing from 12 to 16 carbon atoms or an amine chosen from linear alkylamines, branched alkylamines, cycloalkylamines, alkoxyamines, amino alcohols, cyclic amines containing at least one nitrogen atom in a cyclic structure, an alkylenediamine, a polyetherdiamine or a polyalkylenepolyamine, so as to obtain an auxiliary that is incorporated into the composition of detergents; or
with an alkylene oxide, a phenylene oxide or to a reaction with an alkylene oxide, a phenylene oxide and a glycidyl ether or to a homopolymerization reaction, so as to obtain an epichlorohydrin elastomer; or
of oligomerisation, of co-oligomerisation, of condensation, of dehydrochlorination and of hydrolysis, with water, or with a di- or polyhydroxylated compound which may optionally be halogenated and/or have ether oxide bonds and/or double bonds capable of being halogenated in a subsequent stage, so as to obtain halogenated polyethers-polyols
to a reaction with water, so as to obtain monochloropropanediol.

In the process according to the invention, said collected aqueous effluent contains at least one compound other than water.

The compound may originate from the process according to the invention and/or outside of the process according to the invention.

In the first case, it may be, for example, a compound selected from the group consisting of raw materials used (reactants, catalysts, diluent, solvent), intermediates, products, by-products formed in the process for manufacturing the chemical, and mixtures of at least two of them.

In the second case, it may be, for example, a compound resulting from treatments intended to make the water suitable for a food or industrial use. In this second case, it may also be a compound resulting from treatments intended to reduce the corrosivity of the water and make it suitable for the use in the heating and/or cooling circuits of the plant. In this second case, it may further be an auxiliary compound added to the plant and necessary for its operation, such as lubricants for rotating machines, lubricants for formations of assemblies, and the components present in thermostatting fluids. The lubricants may be organic or mineral lubricants. The lubricants may be of natural origin such as lubricants based on oils and greases, of animal origin and/or of plant origin. The lubricants may also be synthetic lubricants such as silicone oils, for example.

The compound usually originates from the process according to the invention.

The compound is found in said collected aqueous effluent possibly following contact of the waters with the walls of the plant that are contaminated by such compounds. This contamination of the walls may have any normal or abnormal origin. An example of normal origin is the deposition of compounds on the walls of a reactor or of a separation column during the process for manufacturing the chemical. These compounds are then entrained in the plant washing waters during a shutdown of the manufacturing process. An example of abnormal origin is the piercing of a pipe for transferring a reaction medium, with contamination of the outer surface of the pipe.

The compound is found in said collected aqueous effluent also possibly following contact of the waters with such compounds but without any contact of such waters with the walls of the plant. Quenching of a gaseous or liquid compound resulting from a leakage and no longer in contact with the wall of the plant, by rain water for instance, is an example of such contact.

The compound may also originate from overhead waters, surface waters, ground waters, waters from drinking water distribution networks, waters from industrial water distribution networks, plant cleaning waters, waters used for heating and cooling the plants. Surface waters may for example contain metallic salts like sodium, potassium, calcium, magnesium salts and any mixture of at least two of them. These salts are usually found as chloride, bicarbonate, nitrate, sulfate any mixture of at least two of them. Overhead waters may for example contain acidic compounds like for instance sulfuric acid, hydrogen chloride, nitric acid, and any mixture of at least two of them. It has surprisingly been found that such compounds do not affect the process when recycled in the process.

The compound present in said collected aqueous effluent exhibits at least one of the following features, a water solubility at 25° C. greater than or equal to 0.01 g/kg of water and a boiling point at 1013 mbar, greater than or equal to −100° C.

The compound present in said collected aqueous effluent has a water solubility at 25° C. that is, often greater than or equal to 0.02 g/kg, frequently greater than or equal to 0.03 g/kg, more often greater than or equal to 0.05 g/kg, more frequently greater than or equal to 0.08 g/kg, in particular greater than or equal to 0.1 g/kg, yet more often greater than or equal to 10 g/kg, yet more frequently greater than or equal to 50 g/kg, still more often greater than or equal to 100 g/kg, still more frequently greater than or equal to 100 g/kg, particularly greater than or equal to 150 g/kg, more particularly greater than or equal to 200 g/kg and yet more particularly greater than or equal to 500 g/kg. In certain cases, the compound may be miscible with water in any proportion.

The compound present in said collected aqueous effluent has a boiling point at 1013 mbar that is often greater than or equal to −50° C., frequently greater than or equal to −10° C., more often greater than or equal to 0° C., more frequently higher than or equal to 25° C., yet more often greater than or equal to 30° C., yet frequently greater than or equal to 35° C., still more often greater than or equal to 40° C., still more frequently greater than or equal to 45° C., in particular greater than or equal to 50° C., more particularly greater than or equal to 100° C., yet more particularly greater than or equal to 150° C., still more particularly greater than or equal to 200° C., specifically greater than or equal to 225° C., more specifically greater than or equal to 250° C. and yet more specifically greater than or equal to 280° C. By boiling point at 1013 mbar one intends to denote the temperature at which the pure compound exhibits a vapour pressure of 1013 mbar.

The compound present in said collected aqueous effluent exhibits often a water solubility at 25° C. greater than or equal to 0.01 g/kg of water and a boiling point at 1013 mbar, greater than or equal to −100° C. The compound present in said collected aqueous effluent exhibits frequently a water solubility at 25° C. greater than or equal to 0.1 g/kg of water and a boiling point at 1013 mbar, greater than or equal to 25° C.

If the compound present in said collected aqueous effluent exists in the critical state, it exhibits usually a critical temperature greater than or equal to 0° C., often greater than or equal to a 35° C. and frequently greater than or equal to 50° C.

The compound present in said collected aqueous effluent usually does not form an azeotropic mixture with water at the pressure of 1013 mbar. The compound present in said collected aqueous effluent preferably does not form an azeotropic mixture with water at 1013 mbar.

The compound present in said collected aqueous effluent may form an azeotropic mixture with water at the pressure of 1013 mbar. The azeotropic mixture may be a maximum boiling temperature azeotropic mixture or a minimum boiling temperature azeotropic mixture. The azeotropic mixture is preferably a maximum boiling temperature azeotropic mixture with water.

The compound is often a compound that can undergo a hydrolysis reaction under the collection and storage conditions of said waters. By hydrolysis reaction, one intends to denote a chemical reaction in which water reacts with the compound to form one or more new substances.

The content of this compound in said collected aqueous effluent is generally greater than or equal to 0.01 g of compound per kg of collected aquoes effluent, usually greater than or equal to 0.03 g/kg, commonly greater than or equal to 0.05 g/kg, in a lot of cases greater than or equal to 0.05 g/kg, sometimes greater than or equal to 0.1 g/kg, frequently greater than or equal to 1 g/kg and often greater than or equal to 5 g/kg. This content is generally less than or equal to 500 g of compound per kg of collected aqueous effluent, usually less than or equal to 300 g/kg, commonly less than or equal to 100 g/kg, in a lot of cases less than or equal to 50 g/kg, sometimes less than or equal to 20 g/kg and frequently less than or equal to 10 g/kg.

The compound present in said collected aqueous effluent may be an organic compound, an inorganic compound, or a mixture of the two. The expression “inorganic compound” is understood to mean a compound whose molecule does not contain a carbon-carbon bond or a carbon-hydrogen bond. The expression “organic compound” is understood to mean a compound whose molecule contains at least one carbon-carbon bond or one carbon-hydrogen bond.

The compound is often an organic compound. The organic compound may be a chemical as defined above.

In the process for manufacturing a chemical according to the invention, the compound is generally chosen from the group consisting of olefins, epoxides, diols, diols derivatives, ethers, esters, aldehydes, ketones, such as acrolein, alcohols, linear, branched or cyclic, aliphatic or aromatic, saturated or unsaturated hydrocarbons, halogenated derivatives of these hydrocarbons, polyhydroxylated aliphatic hydrocarbons, polyhydroxylated aliphatic hydrocarbon esters, carboxylic acids, carboxylic acid esters, other compounds that combine several chemical functions in their molecule, such as chlorohydrins, chlorohydrin esters, partially chlorinated and/or esterified polyhydroxylated aliphatic hydrocarbon oligomers, chloroethers, halogenated alcohols, chlorinated polyols, chloroketones, salts, mineral acids, basic compounds, and mixtures of at least two of them.

In the process according to the invention, the compound is often chosen from the group consisting of olefins, polyhydroxylated aliphatic hydrocarbons, polyhydroxylated aliphatic hydrocarbon esters, carboxylic acids, carboxylic acid esters, chlorohydrins, chlorohydrin esters, partially chlorinated and/or esterified polyhydroxylated aliphatic hydrocarbon oligomers, salts, mineral acids, basic compounds, epoxides, and mixtures of at least two of them. These compounds are often encountered when the process according to the invention is a process for manufacturing a chemical selected from the group consisting of chlorohydrins, epoxides, diols, diols derivatives, epoxy derivatives, and any combination of at least two of those processes. They are frequently encountered when the process according to the invention is a process for manufacturing a chemical selected from the group consisting chlorohydrins, epoxides, epoxy derivatives, and any combination of at least two of those processes.

In the process according to the invention, the compound is often selected from the group consisting of polyhydroxylated aliphatic hydrocarbons, polyhydroxylated aliphatic hydrocarbon esters, carboxylic acids, carboxylic acid esters, chlorohydrins, chlorohydrin esters, partially chlorinated and/or esterified polyhydroxylated aliphatic hydrocarbon oligomers, epoxides, and any mixture of at least two of them.

In the process according to the invention, the compound is more often chosen from the group consisting of polyhydroxylated aliphatic hydrocarbons, polyhydroxylated aliphatic hydrocarbon esters, carboxylic acids, carboxylic acid esters, partially chlorinated and/or esterified polyhydroxylated aliphatic hydrocarbon oligomers, and any mixture of at least two of them.

In the process according to the invention, the compound is more frequently chosen from the group consisting of polyhydroxylated aliphatic hydrocarbons, carboxylic acids, partially chlorinated and/or esterified polyhydroxylated aliphatic hydrocarbon oligomers, and any mixture of at least two of them.

In the process according to the invention, the compound is particularly chosen from the group consisting of polyhydroxylated aliphatic hydrocarbons, partially chlorinated and/or esterified polyhydroxylated aliphatic hydrocarbon oligomers, and any mixture of at least two of them.

In the process according to the invention, the compound is more particularly chosen from the group consisting of polyhydroxylated aliphatic hydrocarbons.

The expression “olefin” is used here to describe a compound having at least one carbon-carbon double bond. Generally, the compound may contain atoms other than carbon atoms, such as hydrogen and halogen atoms. The olefins often encountered are ethylene, propylene, allyl chloride and mixtures of at least two of them. Propylene and allyl chloride are frequently encountered and allyl chloride is more often encountered.

The expression “polyhydroxylated aliphatic hydrocarbon” relates to a hydrocarbon that contains at least two hydroxyl groups attached to two different saturated carbon atoms. The polyhydroxylated aliphatic hydrocarbon may contain, but is not limited to, 2 to 60 carbon atoms.

Each of the carbons of a polyhydroxylated aliphatic hydrocarbon bearing the functional hydroxyl (OH) group cannot possess more than one OH group, and must be of sp3 hybridization. The carbon atom bearing the OH group may be primary, secondary or tertiary. The polyhydroxylated aliphatic hydrocarbon must contain at least two carbon atoms of sp3 hybridization bearing an OH group. The polyhydroxylated aliphatic hydrocarbon includes any hydrocarbon that contains a vicinal diol (1,2-diol) or a vicinal triol (1,2,3-triol) including higher orders of these vicinal or adjacent repeat units. The definition of the polyhydroxylated aliphatic hydrocarbon also includes, for example, one or more 1,3-, 1,4-, 1,5- and 1,6-diol functional groups. The polyhydroxylated aliphatic hydrocarbon may also be a polymer such as polyvinyl alcohol. Geminal diols, for example, are excluded from this class of polyhydroxylated aliphatic hydrocarbons.

The polyhydroxylated aliphatic hydrocarbons may contain aromatic entities or heteroatoms including, for example, heteroatoms of halogen, sulphur, phosphorus, nitrogen, oxygen, silicon and boron type, and mixtures thereof.

Polyhydroxylated aliphatic hydrocarbons include, for example, 1,2-ethanediol (ethylene glycol), 1,2-propanediol (propylene glycol), 1,3-propanediol, 1-chloro-2,3-propanediol(chloropropanediol), 2-chloro-1,3-propanediol(chloropropanediol), 1,4-butanediol, 1,5-pentanediol, cyclohexanediols, 1,2-butanediol, 1,2-cyclohexanedimethanol, 1,2,3-propanetriol (also known as “glycerol” or “glycerine”), and mixtures thereof. Often, the polyhydroxylated aliphatic hydrocarbon includes, for example, 1,2-ethanediol, 1,2-propanediol, 1,3-propanediol, chloropropanediol and 1,2,3-propanetriol, and mixtures of at least two of them. Frequently, the polyhydroxylated aliphatic hydrocarbon includes, for example, 1,2-ethanediol, 1,2-propanediol, 2-chloro-1,3-propanediol, 3-chloro-1,2-propanediol and 1,2,3-propanetriol, and mixtures of at least two of them. 1,2,3-propanetriol or glycerol is more particularly encountered.

The polyhydroxylated aliphatic hydrocarbon may be obtained from fossil raw materials or from renewable raw materials, preferably from renewable raw materials, as defined in International Application WO 2005/054167 by SOLVAY SA, more specifically from page 1, line 26 to page 4, line 2, in International Application WO 2006/100312 by SOLVAY SA, more specifically from page 3, line 29 to page 5, line 24, and in French Application FR 07/56125 by SOLVAY SA, more specifically from page 4, line 35 to page 5, line 22, the contents of which are incorporated herein by reference. In particular, the polyhydroxylated aliphatic hydrocarbon may be obtained from renewable raw materials via any process such as, for example, in processes for conversion of oils and/or fats of animal or plant origin, such as hydrolysis, saponification, transesterification, aminolysis and hydrogenation processes and enzymatic rupture processes. The polyhydroxylated aliphatic hydrocarbon may also be obtained in processes for conversion of monosaccharides and polysaccharides and derived alcohols, such as fermentation processes and thermochemical processes, for instance hydrogenation and hydrogenolysis.

The expression “chlorohydrin” is used here to describe a compound containing at least one hydroxyl group and at least one chlorine atom attached to various saturated carbon atoms. A chlorhydrin that contains at least two hydroxyl groups is also a polyhydroxylated aliphatic hydrocarbon. Chlorohydrins that are often encountered are chloroethanol, chloropropanol, chloropropanediol, dichloropropanol and mixtures of at least two of them. Dichloropropanol is particularly encountered. Chlorohydrins that are frequently encountered are 2-chloroethanol, 1-chloropropane-2-ol, 2-chloropropane-1-ol, 1-chloropropane-3-ol, 1-chloropropane-2,3-diol, 2-chloropropane-1,3-diol, 1,3-dichloropropane-2-ol, 2,3-dichloropropane-1-ol and mixtures of at least two of them. 1-chloropropane-2,3-diol, 2-chloropropane-1,3-diol, 1,3-dichloropropane-2-ol, 2,3-dichloropropane-1-ol and mixtures of at least two of them are specifically encountered. 1,3-dichloropropane-2-ol, 2,3-dichloropropane-1-ol and mixtures thereof are more specifically encountered.

The expression “epoxide” is used here to describe a compound having at least one oxygen atom bridged over a carbon-carbon bond. Generally, the carbon atoms of the carbon-carbon bond are adjacent and the compound may contain atoms other than carbon and oxygen atoms, such as hydrogen and halogen atoms. The epoxides often encountered are ethylene oxide, propylene oxide, glycidol and epichlorohydrin. Epichlorohydrin is frequently encountered.

The carboxylic acids are generally monocarboxylic or polycarboxylic acids containing 1 to 20 carbon atoms. These acids may be used as catalysts in the process for manufacturing the chemical. When the chemical is a chlorohydrin, these carboxylic acids are as described in International Application WO 05/054167 by SOLVAY SA, more specifically from page 6, line 28, to page 7, line 35, the content of which is incorporated herein by reference.

The carboxylic acid esters are generally esters of the monocarboxylic or dicarboxylic acids mentioned above with the polyhydroxylated aliphatic hydrocarbons and/or the chlorohydrins mentioned above.

The mineral acids are usually hydrogen halides and frequently hydrogen chloride.

In the process according to the invention, the salts may be chosen from alkali or alkaline-earth metal chlorides, nitrates, sulphates, hydrogensulphates, hydroxides, carbonates, hydrogencarbonates, phosphates, hydrogenphosphates and borates, and mixtures of at least two of them. Alkali and alkaline-earth metal chlorides are usually encountered.

In the process according to the invention, the basic compound may be an organic or inorganic basic compound. Organic basic compounds are for example amines, phosphines and ammonium, phosphonium or arsonium hydroxides. Inorganic basic compounds are preferred. The inorganic basic compound may be chosen from alkali or alkaline-earth metal oxides, hydroxides, carbonates, hydrogencarbonates, phosphates, hydrogenphosphates and borates, ammonia and mixtures of at least two of them. Alkali and alkaline-earth metal oxides and hydroxides are usually encountered.

The partially chlorinated and/or esterified polyhydroxylated aliphatic hydrocarbon oligomers are usually partially chlorinated and/or esterified glycerol oligomers.

In a first embodiment of the process according to the invention, the chemical manufactured is dichloropropanol. In this embodiment, dichloropropanol is usually obtained by reacting glycerol with a chlorinating agent, preferably comprising hydrogen chloride.

In this embodiment, said collected aqueous effluents generally contain dichloropropanol, glycerol, chloropropanediol, alone or as a mixture. These compounds are difficult to separate from water by stripping. They contribute to the chemical oxygen demand (COD) of said waters. They are generally present in a low concentration which makes said waters not very suited to treatment in a biological treatment plant.

In a first variant of the first embodiment, the process for manufacturing dichloropropanol comprises the following steps:
(a) glycerol is subjected to a reaction with a chlorinating agent comprising hydrogen chloride, so as to obtain at least dichloropropanol and water, in a liquid reaction medium in equilibrium with a gas phase;
(b) at least one first portion of the liquid reaction medium and/or of the gas phase from step (a) is subjected to a distillation and/or steam distillation and/or stripping operation, so as to separate at least one mixture comprising dichloropropanol, hydrogen chloride and water.

In a first aspect of the first variant of the first embodiment, at least one portion of said collected aqueous effluent is recycled to step (a) of the process for manufacturing dichloropropanol. This recycling has the following advantages:
substitution of at least one portion of the water needed for the preparation of the chlorinating agent, when this is partly composed of aqueous hydrogen chloride;
conversion of at least one portion of the COD to a reusable product, for example conversion of glycerol and/or of monochloropropanediol to dichloropropanol.

In a second aspect of the first variant of the first embodiment, at least one portion of said collected aqueous effluent is recycled to step (b) of the process for manufacturing dichloropropanol.

In a third aspect of the first variant of the first embodiment, at least one first portion of said collected aqueous effluent is recycled to step (a) and at least one second portion of said collected aqueous effluent is recycled to step (b) of the process for manufacturing dichloropropanol.

A second variant of the first embodiment of the process according to the invention is the same as the first variant except that the process for manufacturing dichloropropanol comprises a supplementary step (c) in which:
(c) at least one second portion of the liquid reaction medium and/or of the gas phase from step (a) is subjected to a high-temperature oxidation operation so as to generate at least hydrogen chloride and water.

The first three aspects of the second variant of the first embodiment are the same as the three aspects of the first variant.

In a fourth aspect of the second variant of the first embodiment, at least one portion of said collected aqueous effluent is recycled to step (c) of the process for manufacturing dichloropropanol. This recycling has the advantage of reusing the hydrocarbon-based content of said waters in the form of energy and of recovering the chlorocarbon-based content of said waters in the form of reusable hydrogen chloride.

In other aspects of the second variant of the first embodiment, said collected aqueous effluent are recycled partly to step (a) and partly to step (c), or partly to step (b) and partly to step (c), or partly to step (a), partly to step (b) and partly to step (c), of the process according to the invention.

In that first embodiment, the glycerol generally contains nitrogen compounds, usually at least one nitrogen compound, as described in Application WO 2009/077528 in the name of Solvay SA, from page 1, line 31, to page 3, line 24, and the content of which is incorporated here by reference.

In that first embodiment, the glycerol generally contains diols, usually at least one diol, as described in Application WO 2009/000773 in the name of Solvay SA, from page 1, line 30, to page 3, line 21, and the content of which is incorporated here by reference.

In that first embodiment, the glycerol generally contains glycerol alkyl ethers, usually at least one glycerol alkyl ether, as described in Application WO 2007/144335 in the name of Solvay SA, from page 1, line 33, to page 3, line 25, and the content of which is incorporated here by reference.

In that first embodiment, the glycerol may also comprise monoalcohols, usually at least one monoalcohol, such as the monoalcohols described in Application WO 2007/144335 in the name of Solvay SA, from page 3, lines 26 to 31, and the content of which is incorporated here by reference.

In that first embodiment, the glycerol may also comprise alkyl esters of fatty acids, usually at least one alkyl ester of fatty acids, glycerol esters, generally at least one glycerol ester, and salts, commonly at least one salt, as described in Application WO 2007/144335 in the name of Solvay SA, from page 5, lines 12 to 20.

In that first embodiment, the glycerol may be obtained starting from fossil raw materials or starting from renewable raw materials, preferably starting from renewable raw materials, as described in WO 2005/054167 of SOLVAY SA, the content of which is incorporated herein by reference, and especially the passages from page 1, line 26, to page 4, line 2.

In that first embodiment, the glycerol may also be obtained starting from fossil raw materials or starting from renewable raw materials, preferably starting from renewable raw materials, as described in WO 2009/000773 of SOLVAY SA, the content of which is incorporated herein by reference, and especially the passages at page 10, lines 16 to 23, and at page 11, lines 4 to 25.

In that first embodiment, the glycerol may have an alkali metal and/or alkaline earth metal content as described in WO 2006/100315 of SOLVAY SA, the content of which is incorporated herein by reference, and especially the passages from page 7, line 11, to page 9, line 10.

In that first embodiment, the glycerol may contain elements other than alkali metals and alkaline earth metals as described in WO 2006/100319 of SOLVAY SA, the content of which is incorporated herein by reference, especially the passages from page 2, line 3 to 8, and from page 6, line 20, to page 9, line 14.

In that first embodiment, the glycerol contains generally an amount of heavy compounds other glycerol and whose boiling temperature under a pressure of 1 bar absolute is at least 15° C. greater than the boiling temperature of dichloropropanol as described in WO 2006/1000316 of SOLVAY SA the content of which is incorporated herein by reference, especially the passages from page 15, line 32, to page 17, line 33.

In that first embodiment, the glycerol may contain glycerol oligomers as described in PCT/EP2009/053766 of SOLVAY SA the content of which is incorporated herein by reference, especially the passages from page 1, line 25, to page 6, line 19.

In that first embodiment, the glycerol may be treated as described in FR 0858362 of SOLVAY SA the content of which is incorporated herein by reference, especially the passages from page 1, lines 16 to 23, and from page 6, line 4, to page 11, line 26.

In that first embodiment, the chlorinating agent is as described in Patent Application WO 2005/054167, from page 4, line 32, to page 5, line 18, in the name of Solvay SA, the content of which is incorporated here by reference.

In that first embodiment, the hydrogen chloride is often a gas or a mixture of a gas and an aqueous solution of hydrogen chloride. The hydrogen chloride may at least partially be obtained from processes such as described in WO 2005/054167 of SOLVAY SA, the content of which is incorporated herein by reference, especially the passages from page 4, line 32, to page 5, line 35.

In that first embodiment, the hydrogen chloride may at least partially be obtained from processes such as described in WO 2006/106153 of SOLVAY SA, the content of which is incorporated herein by reference, especially the passages from page 2, line 10, to page 3, line 20, and from page 11, line 1 to page 18, line 29.

In that first embodiment, the hydrogen chloride may at least partially be obtained from processes such as described in WO 2007/144335 of SOLVAY SA, the content of which is incorporated herein by reference, especially the passages from page 12, line 14, to page 14, line 21.

In that first embodiment, the hydrogen chloride may purified such as described in FR 08/56138 of SOLVAY SA, the content of which is incorporated herein by reference, especially the passages from page 2, line 33, to page 16, line 21.

In that first embodiment, the reaction between glycerol and the chlorinating agent the hydrogen chloride may be carried out in a reaction medium such described in WO 2006/106154 of SOLVAY SA, the content of which is incorporated herein by reference, especially the passages from page 14, line 15, to page 17, line 10.

In that first embodiment, the reaction with the chlorinating agent may be carried out in the presence of a catalyst, preferably a carboxylic acid or a carboxylic acid derivative, as described in Patent Application WO 2005/054167, from page 6, line 24, to page 7, line 35 in the name of Solvay SA, the content of which is incorporated here by reference.

In that first embodiment, the reaction with the chlorinating agent may be carried out for a catalyst concentration, at a temperature, at a pressure and at a residence time such described in WO 2005/054167 of SOLVAY SA, the content of which is incorporated herein by reference, especially the passages from page 8, line 1, to page 10, line 10.

In that first embodiment, the reaction with the chlorinating agent may be carried out such described in WO 2007/054505 of SOLVAY SA, the content of which is incorporated herein by reference, especially the passages from page 1, line 24 to page 6, line 18.

In that first embodiment, the process for manufacturing dichloropropanol may be carried in equipments made of or coated with materials which are resistant to the corrosion by the chlorinating agent under the process conditions, such as described in WO 2005/054167 of SOLVAY SA, the content of which is incorporated herein by reference, especially the passages from page 6, lines 3 to 23.

In that first embodiment, the process for manufacturing dichloropropanol may be carried in equipments made of or coated with materials which are resistant to the corrosion by the chlorinating agent under the process conditions, such as described in WO 2006/100317 of SOLVAY SA, the content of which is incorporated herein by reference, especially the passages from page 23, line 22, to page 27, line 25.

In that first embodiment, the process for manufacturing dichloropropanol according to the invention may be carried in equipments made of or coated with materials which are resistant to the corrosion by the chlorinating agent under the process conditions, such as described in WO 2009/043796 of SOLVAY SA, the content of which is incorporated herein by reference, especially the passages from page 1, line 30, to page 9, line 17, and from page 19, line 25, to page 20, line 33.

In that first embodiment, the chlorination reaction may be carried out in the presence of a solvent such described in WO 2005/054167 of SOLVAY SA, the content of which is incorporated herein by reference, especially the passages at page 11, lines 12 to 36.

In that first embodiment, the reaction with the chlorinating agent may be carried out in the presence of a liquid phase comprising heavy compounds other than glycerol such described in WO 2006/100316 of SOLVAY SA, the content of which is incorporated herein by reference, especially the passages at page 2, lines 18 to 25 and from page 15, line 32, to page 17, line 33.

In that first embodiment, the reaction with the chlorinating agent may be carried out under stirring with a stirring system such described in WO 2008/145729 of SOLVAY SA, the content of which is incorporated herein by reference, especially the passages from page 1, line 30, to page 2, line 33, and from page 6, line 22, to page 14, line 31.

In that first embodiment, the reaction with the chlorinating agent may be carried out in a liquid reaction medium such described in WO 2006/106154 of SOLVAY SA, the content of which is incorporated herein by reference, especially the passages from page 1, line 29, to page 2, line 6, and from page 14, line 15, to page 17, line 10.

In that first embodiment, the reaction with the chlorinating agent may be carried out in a reactor the feeding of which is described in WO 2008/107468 of SOLVAY SA, the content of which is incorporated herein by reference, especially the passages from page 1, line 29, to page 4, line 27, and from page 5, line 34, to page 9, line 17.

In that first embodiment, a separation of the dichloropropanol from the other compounds of the reaction mixture may be carried out such described in WO 2005/054167 of SOLVAY SA, the content of which is incorporated herein by reference, especially the passages from page 12, line 1, to page 17, line 20.

In that first embodiment, a separation of the dichloropropanol from the other compounds of the reaction mixture may be carried out according to methods such described in WO 2006/100313 of SOLVAY SA, the content of which is incorporated herein by reference, especially the passages at page 2, lines 1 to 23, and from page 21, line 7, to page 25, line 25.

In that first embodiment, a separation of the dichloropropanol from the other compounds of the reaction mixture may be carried out according to methods such described in WO 2006/100314 of SOLVAY SA, the content of which is incorporated herein by reference, especially the passages at page 2, lines 6 to page 3, line 4, and from page 18, line 33, to page 22, line 29.

In that first embodiment, a separation of the dichloropropanol from the other compounds of the reaction mixture may be carried out according to methods such described in WO 2006/100320 of SOLVAY SA, the content of which is incorporated herein by reference, especially the passages from page 1, line 30, to page 2, line 23, and from page 6, line 25, to page 10, line 28.

In that first embodiment, a separation of the dichloropropanol from the other compounds of the reaction mixture may be carried out according to methods such described in WO 2006/100315 of SOLVAY SA, the content of which is incorporated herein by reference, especially the passages at page 2, lines 3 to 29, and from page 23, line 3, to page 24, line 13.

In that first embodiment, a separation of the dichloropropanol from the other compounds of the reaction mixture may be carried out according to methods such described in WO 2008/110588 of SOLVAY SA, the content of which is incorporated herein by reference, especially the passages from page 1, line 31, to page 27, line 25.

In that first embodiment, the dichloropropanol is generally obtained as a mixture of 1,3-dichloropropan-2-ol and 2,3-dichloropropan-1-ol isomers such described in WO 2006/100319 of SOLVAY SA, the content of which is incorporated herein by reference, especially the passages from page 23, line 34, to page 24, line 29.

In that first embodiment, the dichloropropanol may contain halogenated ketones such described in WO 2006/100311 of SOLVAY SA, the content of which is incorporated herein by reference, especially the passages at page 2, lines 22 to 34, and from page 22, line 8, to page 23, line 35.

In a second embodiment of the process according to the invention, the chemical manufactured is epichlorohydrin. In this embodiment, epichlorohydrin is usually obtained by subjecting dichloropropanol to a dehydrochlorination reaction with a basic agent. The basic agent may be as described above

In this embodiment, said collected aqueous effluent generally contain epichlorohydrin, glycerol, chloropropanediol, dichloropropanol, alone or as a mixture. These compounds are difficult to separate from water by stripping. They contribute to the chemical oxygen demand (COD) of said waters. They are generally present in a low concentration which makes said waters not very suited to treatment in a biological treatment plant.

In a first variant of the second embodiment, the process for manufacturing epichlorohydrin comprises the following steps:
i. dichloropropanol is reacted with at least one basic compound so as to obtain epichlorohydrin and at least one salt, in a liquid reaction medium; and
ii. at least one portion of the liquid reaction medium from step (i) is subjected to a settling operation in which a first fraction containing most of the epichlorohydrin which was contained in the portion of the reaction medium from step (i) before the settling operation is separated from a second fraction containing most of the salt which was contained in the portion of the reaction medium from step (i) before the settling operation.

In a first aspect of the first variant of the second embodiment, at least one portion of said collected aqueous effluent is recycled to step (i) of the process for manufacturing epichlorohydrin. This recycling has, in particular, the following advantages:
substitution of at least one portion of the water needed for the preparation of the basic compound when this is partly composed of an aqueous solution or suspension;
conversion of at least one portion of the COD to reusable products, for example the conversion of monochloropropanediol and/or dichloropropanol to epichlorohdyrin and/or glycidol.

In a second aspect of the first variant of the second embodiment, at least one portion of said collected aqueous effluent is recycled to step (ii) of the process for manufacturing epichlorohydrin. In this aspect, the recycling has the advantage of being able to supply a portion of the water needed for the separation of the fractions during the settling operation of step (ii) of the process for manufacturing epichlorohydrin.

In a third aspect of the first variant of the second embodiment, a first portion of said collected aqueous effluent is recycled to step (i) and a second portion to step (ii) of the process for manufacturing epichlorohydrin. This recycling combines the advantages of the first two aspects.

A second variant of the second embodiment of the process according to the invention is the same as the first variant except that the process for manufacturing epichlorohydrin comprises a supplementary step (iii) in which:
iii. the first fraction separated in step (ii) is subjected to at least one supplementary treatment chosen from dilution, concentration, evaporation, distillation, steam distillation and/or stripping, liquid/liquid extraction and adsorption operations, alone or in combination.

The first three aspects of the second variant of the second embodiment are the same as the three aspects of the first variant.

In a fourth aspect of the second variant of the second embodiment, at least one portion of said collected aqueous effluent is recycled to step (iii) of the process for manufacturing epichlorohydrin. This recycling has the advantage of being able to recover the hydrocarbon-based content of the waters during the various treatment operations.

In other aspects of the second variant of the second embodiment, said collected aqueous effluent are recycled partly to step (i) and partly to step (iii), or partly to step (ii) and partly to step (iii), or partly to step (i), partly to step (ii) and partly to step (iii), of the process according to the invention.

A third variant of the second embodiment of the process according to the invention is the same as the first variant or as the second variant except that the process for manufacturing epichlorohydrin comprises a supplementary step (iv) in which:
iv. the second fraction separated in step (ii) is subjected to at least one purification treatment chosen from dilution, concentration, evaporation, distillation, steam distillation, stripping, liquid/liquid extraction, adsorption, oxidation, reduction, neutralization, complexation and precipitation operations, aerobic bacterial treatments, anaerobic bacterial treatments and enzymatic treatments, alone or in combination.

In various aspects of the third variant of the second embodiment, said collected aqueous effluent are recycled partly to one of steps (i), (ii), (iii), (iv) of the process according to the invention, alone or in combination.

In this third variant of the second embodiment, the oxidation treatment of step (iv) may be as described in WO 2008/152043 in the name of SOLVAY SA, the content of which is hereby incorporated by reference, more specifically the passage from page 21, line 18, to page 26, line 28. More specifically, in the oxidation treatment, the second fraction separated in step (ii) is submitted to a reaction with a chlorinated oxidizing agent selected from the group consisting of molecular chlorine, dichlorine oxide, chlorine dioxide, perchloric, chloric, chlorous and hypochlorous acids and the corresponding salts, perchlorates, chlorates, chlorites and hypochlorites, and mixtures of at least two of them.

The recycling of said aqueous effluent in the process according to the invention may be carried out in continuous or batch mode. The choice of the mode depends on the amount of waters collected and on the collection frequency of these waters.

In that second embodiment, the process for dehydrochlorinating the dichloropropanol may be such as described in WO 2005/054167 in the name of SOLVAY SA, the content of which is hereby incorporated by reference, more specifically the passage from page 19, line 12 to page 22, line 30.

In that second embodiment, the process for dehydrochlorinating the dichloropropanol may be such as described in WO 2006/100311 in the name of SOLVAY SA, the content of which is hereby incorporated by reference, more specifically the passages at page 2, lines 22 to 25, and from page 22, line 28 to page 23, line 35.

In that second embodiment, the process for dehydrochlorinating the dichloropropanol may be such as described in WO 2008/101866 in the name of SOLVAY SA, the content of which is hereby incorporated by reference, more specifically the passage from page 2, line 1 to page 13, line 16.

In that second embodiment, the process for dehydrochlorinating the dichloropropanol may be such as described in WO 2008/152045 in the name of SOLVAY SA, the content of which is hereby incorporated by reference, more specifically the passages from page 9, line 22, to page 13, line 31.

In that second embodiment, the process for dehydrochlorinating the dichloropropanol may be such as described in WO 2008/152043 in the name of SOLVAY SA, the content of which is hereby incorporated by reference, more specifically the passages from page 7, line 35, to page 8, line 25.

In that second embodiment, the process for manufacturing the epichlorohydrin may be integrated in a global scheme for preparing dichloropropanol such as described in WO 2006/106155 in the name of SOLVAY SA, the content of which is hereby incorporated by reference, more specifically the passages at page 2, lines 26 to 31, and from page 22, line 10 to page 23, line 19.

In that second embodiment, the process for dehydrochlorinating the dichloropropanol may also be carried out such as described in WO 2006/100318 in the name of SOLVAY SA, the content of which is hereby incorporated by reference, more specifically the passages at page 2, lines 23 to page 3, line 26, and from page 24, line 17 to page 31, line 18.

In that second embodiment, the process for dehydrochlorinating the dichloropropanol may also comprise a step of treating water effluents such as described in WO 2009/095429 in the name of SOLVAY SA, the content of which is hereby incorporated by reference, more specifically the passages from page 1, line 24, to page 27, line 26.

In a third embodiment of the process according to the invention, the process is an process, preferably integrated, for manufacturing dichloropropanol and epichlorohydrin. In this third embodiment, dichloropropanol is obtained by reacting glycerol with a chlorinating agent, preferably comprising hydrogen chloride, according to the first embodiment and said dichloropropanol is further reacted with a basic agent in order to obtain epichlorohydrin according to the second embodiment.

Different variants of this third embodiment can be obtained by combining any variant of the first embodiment with any variant of the second embodiment.

Different aspects of the variants of this third embodiment can be obtained by combining any aspects of any variant of the first embodiment with any aspects of any variant of the second embodiment.

The invention also relates to a plant for manufacturing a chemical chosen from the group consisting of chlorohydrins, epoxides, diols, diols derivatives, epoxy derivatives and mixtures of at least two of them, the process comprising a step of chemical reaction for manufacturing the chemical and at least one step selected from the group consisting of the steps of storage, supply, removal, transfer, chemical treatment and physical treatment of compounds used or produced in the process for manufacturing the chemical, said plant comprising at least one system for collecting and recycling, in said manufacturing process, at least one aqueous effluent containing at least one selected from the group consisting of overhead waters, surface waters, ground waters, waters from drinking water distribution networks, waters from industrial water distribution networks, plant cleaning waters, waters used for heating and cooling the plants, and said aqueous effluent containing at least one compound other than water, said compound exhibiting at least one of the following features, a water solubility at 25° C. greater than or equal to 0.01 g/kg of water and a boiling point at 1013 mbar, greater than or equal to −100° C.

The collection system comprises at least one bottom slab on which the other constituent elements of the plant are found. The bottom slab generally has a construction in the form of an inverted floor or arch, generally made of concrete. The bottom slab constitutes a leaktight foundation system. The bottom slab has the role, in particular, of collecting the waters described above.

The invert waters of the plant are not concerned by this collecting system. Those invert waters are process waters by opposition to the aqueous effluents of the present invention and they are collected by a specific foundation raft connected to a chemical sewer.

The collection system generally comprises tanks that make it possible to recover and store the waters collected by the bottom slab. These tanks are generally made of concrete.

The plant also comprises the equipment needed for transferring waters from the bottom slab to the storage tanks, and for recycling these waters, such as for example pumps and pipework.

The plant may optionally comprise a roof, sometimes side panels, so as to limit the amount of water collected by the bottom slab.

The invention finally relates to the use, in at least one step of a process for manufacturing a chemical chosen from the group consisting of chlorohydrins, epoxides, diols, diols derivatives, epoxy derivatives and mixtures of at least two of them, in an industrial plant, of at least one portion of an aqueous effluent is generated, this aqueous effluent containing at least one selected from the group consisting of overhead waters, surface waters, ground waters, waters from drinking water distribution networks, waters from industrial water distribution networks, plant cleaning waters, waters used for heating and cooling the plants, and this aqueous effluent containing at least one compound other than water, said compound exhibiting at least one of the following features, a water solubility at 25° C. greater than or equal to 0.01 g/kg of water and a boiling point at 1013 mbar, greater than or equal to −100° C.

### EXAMPLE

### (According to the Invention)

An aqueous composition containing 0.2 g/kg of hydrochloric acid, 1.67 g/kg of chloropropanediol, 0.17 g/kg of glycerol, 0.97 g/kg of dichloropropanol, 0.03 g/kg of adipic acid, 0.47 g/kg of chlorinated diglycerol and 1.09 g/kg of adipate esters of chloropropanediol, glycerol and dichloropropanol, and exhibiting a total organic carbon content of about 1.6 g of/kg has been prepared. This composition has been used to simulate an aqueous effluent containing overhead water contaminated by a leakage in a process for manufacturing dichloropropanol by hydro chlorination of glycerol, which has been collected and recycled in a process for manufacturing epichlorohydrin from said dichloropropanol, according to the third embodiment of the process according to the invention.

292.2 g of that composition have been placed in a 1 liter thermostated glass reactor equipped with a vertical water-cooled condenser, with a polytetrafluoroethylene coated magnetic bar, with a jacket containing a thermocouple and with a pH electrode. A pump was used to inject 1 N hydrochloric acid and another pump was used to inject a 1 N caustic soda aqueous solution, in the reactor.

The aqueous composition has been agitated and heated at 93° C. A total quantity of 195 g of a sodium hypochlorite aqueous solution was added in 4 equal fractions. The duration between each addition was 15 min. The sodium hypochlorite aqueous solution was constituted of 90.0 g/kg of sodium hypochlorite, 8.0 g/kg of caustic soda and 13.0 g/kg of sodium chlorate. The 1 N caustic soda aqueous solution and after the 1N hydrochloric acid have been added to keep the pH value of the reaction mixture between 8.0 and 9.3. The temperature of the reaction medium was maintained between 91° C. and 96° C. during the addition of the hypochlorite solution and for an additional time of 15 min. A total quantity of 12.6 ml of 1 N caustic soda and a total quantity of 31 ml of 1 N hydrochloric acid were finally added to adjust the pH.

The total organic carbon content of the final mixture was 0.21 g/kg.

1. A process for manufacturing a chemical selected from the group consisting of chlorohydrins, epoxides, diols, diols derivatives, epoxy derivatives, and mixtures of at least two thereof, in an industrial plant, the process comprising a step of chemical reaction for manufacturing the chemical and at least one step selected from the group consisting of storage, supply, removal, transfer, chemical treatment and physical treatment of compounds used or produced in the process for manufacturing the chemical, wherein at least one aqueous effluent is generated, this aqueous effluent containing at least one water selected from the group consisting of overhead waters, surface waters, ground waters, waters from drinking water distribution networks, waters from industrial water distribution networks, plant cleaning waters, and waters used for heating and cooling the plant, said aqueous effluent containing at least one compound other than water, said compound exhibiting at least one of the following features, a water solubility at 25° C. greater than or equal to 0.01 g/kg of water and a boiling point at 1013 mbar, greater than or equal to −100° C., wherein at least one part of said generated aqueous effluent is collected, and wherein at least one portion of said collected aqueous effluent is recycled in said manufacturing process.

2. The process according to claim 1, wherein the chemical is the chlorohydrin dichloropropanol.

3. The process according to claim 1, wherein the chemical is the epoxide epichlorohydrin.

4. The process according to claim 1, wherein the chemical is an epoxy derivative selected from the group consisting of epoxy resins, glycidyl ethers, glycidyl esters, glycidyl amides, glycidyl imides, glycidyl amines, products that can be used as coagulants, wet-strength resins, cationization agents, flame retardants, ingredients for detergents, epichlorohydrin elastomers, halogenated polyethers-polyols, monochloropropanediol, and mixture of at least two thereof.

5. The process according to claim 1, wherein at least 5% by weight of said collected aqueous effluent is recycled in said manufacturing process.

6. The process according to claim 1, wherein:
the overhead waters are selected from the group consisting of rain water, snow, hail, and mixtures of at least two thereof;
the surface waters are selected from the group consisting of water from land ice, water from sea ice, water from snow on the surface of the ground, water from lakes, water from ponds, water from pools, water from streams, water from rivers, water from brooks, water from biological water treatment plants, sea waters, ocean waters, and mixtures of at least two thereof; and
the ground waters are selected from the group consisting of waters from springs, waters from water tables, waters from underground streams, and mixtures of at least two thereof.

7. The process according to claim 1, wherein the waters are in the liquid state or in the gas state, or in a combination of the liquid state and of the gas state.

8. The process according to claim 1, wherein said compound exhibits a critical temperature greater than or equal to 0° C.

9. The process according to claim 1, wherein the content of the compound per kg of said collected aqueous effluent is greater than or equal to 0.01 g and less than or equal to 500 g.

10. The process according to claim 1, wherein the compound is selected from the group consisting of olefins, epoxides, diols, diols derivatives, epoxy derivatives, ethers, esters, aldehydes, ketones, alcohols, linear, branched or cyclic, aliphatic or aromatic, saturated or unsaturated hydrocarbons, polyhydroxylated aliphatic hydrocarbons, polyhydroxylated aliphatic hydrocarbon esters, carboxylic acids, carboxylic acid esters, halogenated derivatives of these hydrocarbons, chlorohydrins, chlorohydrin esters, partially chlorinated and/or esterified polyhydroxylated aliphatic hydrocarbon oligomers, chloroethers, halogenated alcohols, chlorinated polyols, chloroketones, salts, mineral acids, basic compounds, and mixtures of at least two thereof.

11. The process according to claim 1, wherein the chemical is the chlorohydrin dichloropropanol, and wherein said process comprises the following steps:
(a) glycerol is subjected to a reaction with a chlorinating agent comprising hydrogen chloride, so as to obtain at least dichloropropanol and water, in a liquid reaction medium in equilibrium with a gas phase; and
(b) at least one first portion of the liquid reaction medium and/or of the gas phase from step (a) is subjected to a distillation and/or steam distillation, and/or stripping operation, so as to separate at least one mixture comprising dichloropropanol and water,
and wherein at least one portion of said collected aqueous effluent is recycled in at least one of the steps (a) and (b) of the process for manufacturing dichloropropanol.

12. The process according to claim 11, comprising a supplementary step (c) in which:
(c) a second portion of the liquid reaction medium and/or of the gas phase from step (a) is subjected to a high-temperature oxidation operation so as to generate at least hydrogen chloride and water,
and wherein at least one portion of said collected aqueous effluent is recycled to step (c) of the process for manufacturing dichloropropanol.

13. The process according to claim 1, wherein the chemical is the epoxide epichlorohydrin, and wherein said process comprises the following steps:
i. dichloropropanol is reacted with at least one basic compound so as to obtain epichlorohydrin and at least one salt, in a liquid reaction medium; and
ii. at least one portion of the liquid reaction medium from step (i) is subjected to a settling operation in which a first fraction containing most of the epichlorohydrin which was contained in the portion of the reaction medium from step (i) before the settling operation is separated from a second fraction containing most of the salt which was contained in the portion of the reaction medium from step (i) before the settling operation,
and wherein at least one portion of said collected aqueous effluent is recycled to at least one of the steps (i) and (ii) of the process for manufacturing epichlorohydrin.

14. The process according to claim 13, comprising a supplementary step (iii) in which:
iii. the first fraction separated in step (ii) is subjected to at least one supplementary treatment selected from the group consisting of dilution, concentration, evaporation, distillation, steam distillation, stripping, liquid/liquid extraction, and adsorption operations, alone or in combination,
and wherein at least one portion of said collected aqueous effluent is recycled to step (iii) of the process for manufacturing epichlorohydrin.

15. The process according to claim 13, comprising a supplementary step (iv) in which:
iv. the second fraction separated in step (ii) is subjected to at least one purification treatment selected from the group consisting of dilution, concentration, evaporation, distillation, steam distillation, stripping, liquid/liquid extraction, adsorption, oxidation, reduction, neutralization, complexation and precipitation operations, aerobic bacterial treatments, anaerobic bacterial treatments, and enzymatic treatments, alone or in combination,
and wherein at least one portion of said collected aqueous effluent is recycled to step (iv) of the process for manufacturing epichlorohydrin.

16. The process according to claim 15, wherein in the oxidation treatment, the second fraction separated in step (ii) is submitted to a reaction with a chlorinated oxidizing agent selected from the group consisting of molecular chlorine, dichlorine oxide, chlorine dioxide, perchloric, chloric, chlorous and hypochlorous acids, the corresponding salts, perchlorates, chlorates, chlorites and hypochlorites, and mixtures of at least two thereof.

17. A plant for manufacturing a chemical selected from the group consisting of chlorohydrins, epoxides, diols, diols derivatives, epoxy derivatives, and mixtures of at least two thereof, the process comprising a step of chemical reaction for manufacturing the chemical and at least one step selected from the group consisting of storage, supply, removal, transfer, chemical treatment, and physical treatment of compounds used or produced in the process for manufacturing the chemical, said plant comprising at least one system for collecting and recycling, in said manufacturing process, at least one aqueous effluent containing at least one water selected from the group consisting of overhead waters, surface waters, ground waters, waters from drinking water distribution networks, waters from industrial water distribution networks, plant cleaning waters, waters used for heating and cooling the plant, and said aqueous effluent containing at least one compound other than water, said compound exhibiting at least one of the following features, a water solubility at 25° C. greater than or equal to 0.01 g/kg of water and a boiling point at 1013 mbar, greater than or equal to −100° C.

18. The process according to claim 14, comprising a supplementary step (iv) in which:
iv. the second fraction separated in step (ii) is subjected to at least one purification treatment selected from the group consisting of dilution, concentration, evaporation, distillation, steam distillation, stripping, liquid/liquid extraction, adsorption, oxidation, reduction, neutralization, complexation and precipitation operations, aerobic bacterial treatments, anaerobic bacterial treatments, and enzymatic treatments, alone or in combination,
and wherein at least one portion of said collected aqueous effluent is recycled to step (iv) of the process for manufacturing epichlorohydrin.

19. The process according to claim 18, wherein in the oxidation treatment, the second fraction separated in step (ii) is submitted to a reaction with a chlorinated oxidizing agent selected from the group consisting of molecular chlorine, dichlorine oxide, chlorine dioxide, perchloric, chloric, chlorous and hypochlorous acids, the corresponding salts, perchlorates, chlorates, chlorites and hypochlorites, and mixtures of at least two thereof.
